(19) United States
(12) Patent Application Publication
(10) Pub. No.: US 2025/0032660 A1
(43) Pub. Date: Jan. 30, 2025

(54) USE OF A TEXTILE ARTICLE CONTAINING GRAPHENE TO PRODUCE A BANDAGE FOR WOUNDS

(71) Applicant: DIRECTA PLUS S.P.A., Lomazzo (CO) (IT)

(72) Inventors: Laura Giorgia Rizzi, Saronno (IT); Giulio Giuseppe Cesareo, Como (IT)

(21) Appl. No.: 18/716,544

(22) PCT Filed: Dec. 15, 2022
(86) PCT No.: PCT/EP2022/086188

§ 371 Date: Jun. 5, 2024

(30) Foreign Application Priority Data
Dec. 17, 2021 (IT) 102021000031709

(51) Int. Cl. A61L 15/18; A61L 15/44

(52) U.S. Cl. CPC A61L 15/18; A61L 15/44; A61L 2300/216; A61L 2300/404; A61L 2400/12

(57) ABSTRACT

Use of a textile article containing graphene to produce a bandage to be applied on wounds, wherein said graphene comprises graphene nano-platelets in which at least 90% has a lateral dimension (x, y) from 500 to 50000 nm and a thickness (z) from 0.34 to 50 nm, and wherein said textile article comprises from 0.01 to 20 g of said graphene per square meter.

The present invention relates to the use of a textile article containing graphene to produce a bandage for treating wounds.

### BACKGROUND OF THE INVENTION

The use of dressings, bandages and/or adhesive plasters for application on wounds is known in order to prevent or treat infections and promote cicatrization of the skin and healing of the wound. For this purpose bandages, dressings and/or adhesive plasters, which are often medicated, i.e., treated with disinfectant and optionally cicatrizing substances, or substances in any case having therapeutic properties for the wound, are commonly used.

CN108815553A describes a self-adhesive graphene bandage. The bandage is composed of a substrate of graphene elastic fabric and of a graphene glue. The elastic fabric consists of fibers obtained by spinning a composite material of graphene-polymer and cotton fibers. The graphene glue is composed of from 80 to 90 parts of natural latex, from 3 to 5 parts of a leveling agent and from 0.01 to 10 parts of graphene.

CN108113800A describes an elastic bandage comprising one elastic base layer. Each side of the elastic base layer is provided with a layer composed of a polypropylene film. The outer side of each layer of polypropylene film is provided with an antibacterial layer of graphene on which nano-particles of silver are adsorbed. A layer of bamboo fiber fabric is arranged between each antibacterial layer and the corresponding layer of polypropylene film. The elastic base layer, the layers of polypropylene film and the antibacterial layers are joined together by a medical grade heat melt glue. The surface of the elastic bandage is provided with a plurality of protrusions, adapted to form a space between the elastic bandage and the skin, so as to improve the breathability of the bandage.

CN201710939963A describes a new method of disinfecting a wound through a gold nanoclusters-graphene (AuNCs-Go) composite and an adhesive bandage based on ascorbic acid (AA).

As can be seen from the brief description provided above, these known bandages are particularly complex, both as regards structure and production method.

For example, the need to have fibers containing graphene and to combine them with cotton fibers to obtain a fabric that must then be treated with a graphene glue makes the bandage of CN108815553A particularly complex.

With regard to the solutions proposed in CN108113800A and CN201710939963A, they require rare and expensive substances, such as silver and gold, to be associated with the graphene.

PL 238 131 B1 discloses a potentiometric pH sensor to measure the pH value directly in the vicinity of a wound. The pH sensor contains an electrode of polyurethane and ruthenium oxide with the addition of graphene nanoflakes in a polymer support, and a reference electrode containing polyurethane, metallic silver and silver chloride. Both the indicator electrode and the reference electrode are applied to a single fiber acceptable for use in medical dressings. The single fibers to which the electrodes are applied are in particular made of polyester, cotton, cotton-polyester, cotton-polyamide. The graphene nanoflakes have a diameter of 20 to 30 μm. The sensor electrodes may be used directly as monofilaments or may be integrated into a fabric by weaving. PL 238 131 B1 requires rare and expensive substances, such as silver and ruthenium, and does not deal with bandages for healing wounds but deals with pH sensor to control the state of a wound by measuring the pH.

WO 2018/202747 A1 discloses a polyurethane film comprising graphene and its preparation process. It does mention using the polyurethane film to make bandages for treating wounds. Rezaei Marjan et al: “Nano-curcumin/graphene platelets loaded on sodium alginate/polyvinyl alcohol fibers as potential wound dressing”, JOURNAL OF APPLIED POLYMER SCIENCE, vol. 138, no. 35, 19 Apr. 2021. p 50884, discloses a wound dressing nanocomposite made by electrospinning of sodium alginate, polyvinyl alcohol and graphene nanoplatelets followed by crosslinking the nanofibers by thermal treatment and ionic bonding. The crosslinked fibers are then loaded by micles/polymerosomes containing curcumin. The fabricated composite is obtained by a complex process which increases costs. No information on the size and purity of the graphene used is provided. Therefore, it would be desirable to be able to obtain a graphene bandage that is effective in treating wounds, but which is at the same time relatively simple both as regards structure and production process.

### SUMMARY OF THE INVENTION

Therefore, an object of the present invention is to provide a bandage comprising graphene that can advantageously be used for protecting, disinfecting and treating wounds.

Another object of the present invention is to provide a bandage of this type that is self-adhesive.

Therefore, an aspect of the present invention relates to the use of a textile article containing graphene to produce a bandage to be applied on wounds, wherein said graphene comprises graphene nano-platelets in which at least 90% has a lateral dimension (x, y) from 500 to 50000 nm and a thickness (z) from 0.34 to 50 nm, and said textile article comprises from 0.01 to 20 g of said graphene per square meter.

According to another aspect, the C/O ratio of the graphene is ≥30:1.

In an embodiment of the invention, the textile article consists of a polymeric film in which the graphene is dispersed.

In another embodiment of the invention, the textile article comprises:
a textile substrate selected from a polymeric film, a fabric and a nonwoven, and
graphene, or a composition comprising graphene and a binding agent selected from a synthetic polymer and a polysaccharide.

According to another aspect, the invention relates to the use of a textile article containing graphene to produce a bandage to be applied on wounds, as defined above, wherein said composition comprises, in addition to the binding agent and to the graphene, a natural phytotherapeutic substance with antimicrobial and antiviral properties.

According to yet another aspect, the invention relates to the use of a textile article containing graphene to produce a bandage to be applied on wounds, as defined above, in which said bandage is self-adhesive.

### BRIEF DESCRIPTION OF THE DRAWINGS

FIGS. 1-3 show embodiments of bandages for wounds containing graphene according to the invention;

### DESCRIPTION OF THE INVENTION

The textile article for producing a bandage for wounds according to the present invention comprises a textile substrate selected from a polymeric film, a fabric and a nonwoven. This textile article is suitable for producing a bandage for treating wounds.

In the present description, the terms “bandage” and “dressing” are used interchangeably, as are the terms “adhesive bandage”, “adhesive dressing” and “adhesive plaster”.

In the present description, the term “comprising” includes the term “consisting of”.

According to the present invention the term “textile substrate” is meant as a substrate as defined above, on which graphene or a composition comprising graphene has not yet been applied. Instead, the term “textile article” is meant as the textile substrate on which graphene or a composition containing graphene, optionally dispersed in a binding agent, has been applied.

In the present description, the term “applied” referred to the graphene, or the composition containing graphene, includes various methods of application to the textile substrate, such as dispersion within the substrate, printing with full coating on the substrate, impregnation of the substrate and spray coating of the substrate.

The term “nonwoven” indicates a substantially flat textile substrate obtained with processes other than weaving. In the nonwoven the fibers have a random arrangement, without the formation of a structure arranged in weft and warp threads, and are typically arranged in layers or crisscrossed and are bonded together mechanically, with adhesives or with heat processes.

The nonwovens used fall within the following categories:

### Spunlace

This is a nonwoven deriving from a process called hydroentangling. The process uses high pressure water jets that perforate the fabric and entangle the fibers giving the fabric greater substance. Consolidation of plies of fibers by means of high pressure water jets causes these jets to perforate the fabric and entangle the fibers without damaging them, as can occur with needle punching. Entangling of the fibers in various directions gives the nonwoven isotropic properties and the same strength in various directions.

### Spunbond

This is a nonwoven that is obtained by processing nonwoven synthetic fibers. The feature of this nonwoven is that of thermal point bonding of the fibers. This feature mechanically bonds the fibers to one another and imparts the “point bonding” feature, which is usually square or oval and makes the fabric soft and at the same time strong.

With regard to woven fabric, which is instead obtained with normal weaving processes, it can be made of natural, artificial or synthetic fibers. With regard to the nonwoven, it is typically made of artificial or synthetic fibers.

Useful natural fibers include, for example, wool, silk and cotton. Useful artificial fibers include modified or regenerated cellulose fibers, such as viscose and cellulose acetate. Useful synthetic fibers comprise polyamide, including aromatic polyamides (aramids), polyester, polyurethane, polyacrylonitrile, polycarbonate, polypropylene, polyvinyl chlorine and their blends. Moreover, fabrics obtained from blends of natural, artificial and synthetic fibers can advantageously be used.

The term “polymeric film” is meant as a film made of synthetic resin, according to the customary processing technologies of plastic materials. According to a current terminology, the polymeric film is at times referred to as “polymeric membrane”.

As a function of the specific application, the bandage can have various structures. For example, it can be open or folded, and can have various shapes and sizes according to the part of the body on which it is to be applied.

The textile article for producing the bandage according to the invention comprises from 2 to 20 g of graphene per square meter, and the graphene is in the form of nano-platelets in which at least 90% has a lateral dimension (x, y) from 500 to 50000 nm and a thickness (z) from 0.34 to 50 nm.

According to an aspect of the invention, the graphene contained has a C/O ratio ≥30:1. preferably 40:1, more preferably 50:1, even more preferably 60:1, or 70:1.

According to a preferred aspect of the invention, at least 90% of the graphene nano-platelets has a lateral dimension (x, y) from 100 to 10000 nm.

According to a preferred aspect of the invention, at least 90% of the graphene nano-platelets has a thickness (z) from 0.34 to 10 nm.

According to an aspect of the invention, at least 90% of the graphene nano-platelets has a lateral dimension (x, y) from 100 to 10000 nm, a thickness (z) from 0.34 to 10 nm and a C/O ratio ≥100.

According to an aspect of the invention, the graphene contained in the textile article is present in an amount from 4 to 15 g of graphene per square meter.

The bandage produced with the textile article according to the present invention exhibits an antibacterial, antiviral, antifungal and yeasticidal activity, as will be explained in more detail below.

Where required, the composition for application of the graphene on the textile substrate is preferably in liquid or paste form, where the liquid is preferably water or a mixture of water with other solvents and/or dispersants.

In an embodiment the composition comprises from 0.5 to 20% by weight of graphene with respect to the total weight of the composition, preferably from 1.5 to 15% by weight of graphene, more preferably between 2 and 10% by weight of graphene. As a function of its graphene content, an amount of composition sufficient to deposit from 0.01 to 20 g of graphene per square meter, as defined above, is thus applied on the textile substrate.

With regard to graphene, scientific and patent literature discloses various preparation methods, such as chemical vapor deposition, epitaxial growth, chemical exfoliation and chemical reduction of the oxidized form graphene oxide (GO).

The Applicant Directa Plus S.p.A. is the holder of patents and patent applications relating to graphene production methods, such as EP 2 038 209 B1, WO 2014/135455 A1 and WO 2015/193267 A1. The last two patent publications describe production methods of pristine graphene dispersions, from which it is possible to obtain graphene nano-platelets with the size and with the features required to produce the bandage according to the present invention. According to embodiments described in the patent documents mentioned above by the Applicant Directa Plus S.p.A., the process for producing graphene is a continuous process, carried out by continuously feeding graphite flakes to the high temperature expansion step, continuously discharging the expanded graphite thus obtained in an aqueous medium and continuously subjecting the expanded graphite dispersed in the aqueous medium to exfoliation and size reduction treatment carried out with ultrasonication and/or high pressure homogenization methods.

As described in these patent documents, the final dispersion of graphene nano-platelets obtained can be concentrated or dried, according to the final form desired for the graphene. The object of drying the dispersion is to obtain a dry powder that is easily redispersible in various matrices, both solvents and polymers, where liquid is not desirable or manageable at process level, or where water cannot be used due to chemical incompatibility.

In the present description the size of the graphene nano-platelets is defined with reference to a system of Cartesian axes x, y, z, it being understood that the particles are substantially flat platelets but may also have an irregular shape. In any case, the lateral dimension and the thickness provided with reference to the directions x, y and z must be intended as the maximum dimensions in each of the aforesaid directions.

The lateral dimensions (x, y) of the graphene nano-platelets are determined, within the scope of the production process described above, by direct measurement on the scanning electron microscope (SEM), after having diluted the final dispersion in a ratio of 1:1000 in deionized water and added it dropwise to a silicon oxide substrate placed on a plate heated to 100° C. Alternatively, if nano-platelets in dry state are available, SEM analysis is carried out directly on the powder deposited on a double-sided adhesive carbon disk (carbon tape). In both cases measurement is carried out on at least 100 nano-platelets.

The thickness (2) of the graphene nano-platelets is determined with the atomic force microscope (AFM), which is essentially a profilometer with subnanometer resolution, widely used for characterization (mainly morphological) of surfaces and of nanomaterials. This type of analysis is commonly used to evaluate the thickness of graphene flakes, produced with any method, and therefore to detect the number of layers forming the flake (single layer=0.34 nm).

The thickness (z) can be measured using a dispersion of nano-platelets in a ratio of 1:1000 in isopropanol, from which 20 ml is collected and sonicated in an ultrasonic bath (Elmasonic S40) for 5 minutes. The nano-platelets are then deposited as described for SEM analysis and are scanned directly with an AFM tip, where the measurement provides a topographical image of the graphene flakes and their profile with respect to the substrate, enabling precise measurement of the thickness. The measurement is carried out on at least 50 nano-platelets. Alternatively, if nano-platelets in dry state are available, the powder is dispersed in isopropanol at a concentration of 2 mg/L. 20 ml is collected and sonicated in an ultrasonic bath (Elmasonic S40) for 30 minutes. The nano-platelets are then deposited as described for SEM analysis and are scanned by AFM.

The C/O ratio of the graphene is determined by means of elemental analysis performed by elemental analyzer (CHNS OR), which provides the percentage by weight of the various elements. By normalizing the values obtained with respect to the atomic weight of the C and O species and finding their ratio, the C/O ratio is obtained.

Graphene having the aforesaid characteristics is produced and marketed by the Applicant Directa Plus S.p.A. with the trade name G+®.

In addition to graphene, the composition comprises a binder selected from synthetic polymers and polysaccharides.

When the binder is a synthetic polymer, it is selected from the group consisting of polyurethanes, polyacrylates, polybutadiene and copolymers of acrylic acid.

Among the polyurethanes, anionic polyurethanes are preferred, obtainable for example through reaction of one of more diisocyanates, preferably aliphatic or cycloaliphatic diisocyanates, with one or more polyester diols, and preferably one or more hydroxy carboxylic acids, for example hydroxy acetic acid, or preferably dihydroxy carboxylic acids.

A preferred binder is a polyester based aliphatic polyurethane formulated with isocyanate crosslinker.

In the embodiment with binder consisting of a synthetic polymer, the composition comprises:
a) from 0.5 to 20% by weight of graphene
b) from 10 to 40% by weight, preferably from 10 al 30% by weight, of a polymer binder;
b) from 1 to 10% by weight, preferably 3 to 8% by weight, of a compatibilizing solvent for said polymer binder,
c) from 0.1 to 2% by weight, preferably from 0.15 to 1.5% by weight, of a thickener,
e) from 28 to 88.4% by weight of water.

An example of polymer binder that can be used is water-based pre-catalyzed polyurethane resin containing around 30% of dry polymer, marketed by CPL Chimica, Italy as Resina E9010.

An example of compatibilizing solvent is ethylene glycol.

An example of dispersant consists of naphthalene sulfate.

Examples of inorganic natural thickeners are laminar silicates such as bentonite. Examples of organic natural thickeners are proteins such as casein or polysaccharides. Natural thickeners chosen from agar agar, gum arabic and alginates are particularly preferred.

Examples of synthetic thickeners are generally liquid solutions of synthetic polymers, in particular polyacrylates.

In the embodiment with binder consisting of a polysaccharide, the composition comprises:
a) from 0.5 to 20% by weight of graphene;
b) from 1.0 to 50% by weight of polysaccharide;
c) from 30 to 97.5% of water.

The viscosity of the composition depends on the type of binder and on the application method of the composition on the textile substrate. In general, it is in the range from 800 to 120000 cPs (from 0.8 to 120 Pa·s) and is mainly regulated by the amount of binder and/or thickener. The viscosity is measured according to the standard ISO 2555/1652 using a Fungilab series Viscolead PRO rotational viscometer, R6 spindle, speed rpm 10, T=20° C.

The viscosity of the composition is preferably in the range from 100 to 100000 cPs, or from 10 to 100 Pa·s.

The polyurethane film according to the invention comprises a polyurethane resin and graphene in an amount from 1 to 30% by weight with respect to the total weight of the film. Preferably, the graphene is present in an amount from 2 to 25% by weight with respect to the total weight of the film, more preferably between 3 and 15%.

In the embodiment of the bandage according to the invention, in which the textile article consists of a polymeric film in which the graphene is dispersed, the film consists of a polymer selected from polyurethane, polyester, polyethylene, polypropylene, polyamide. The preferred polymer is polyurethane. The polymeric film can be microperforated, to promote its breathability.

Polymeric films containing graphene are prepared with methods known in the art. A film of this kind consisting of polyurethane is described, for example, in WO 2018/202747 A1.

According to an aspect of the present invention, the composition to be applied on the textile substrate to obtain a textile article containing graphene to produce a bandage to be applied on wounds, as defined above, comprises a natural phytotherapeutic substance with antimicrobial, antiviral, antifungal and yeasticidal properties. Moreover, it was surprisingly found that the specific antiviral action of some natural phytotherapeutic substances in relation to the virus SARS-COV-2 can be added to the antibacterial and antiviral action exerted by the graphene deposited on the textile article.

Therefore, another aspect of the present invention is a bandage for treating wounds that, in addition to graphene, further comprises an effective quantity of natural phytotherapeutic substance selected from the group consisting of curcumin, emodin, α-hederine and thymequinone.

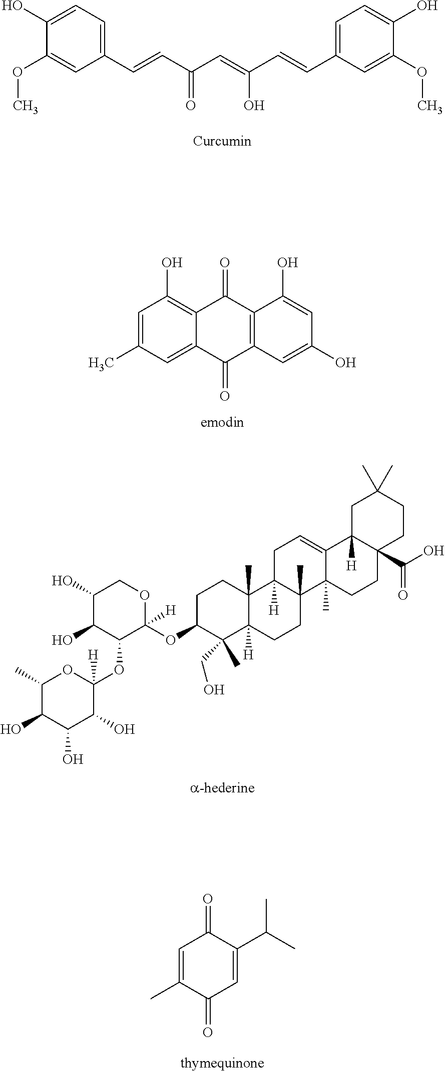

The aforesaid substances were the subject of studies on molecular dynamics and sensitivity to various coronaviruses. In particular, tests were conducted on cytotoxicity and inhibition of SARS-COV-2 infection. The tests proved the effectiveness of curcumin, emodin, α-hederine and thymequinone in the inhibition of Sars COV-2 infection.

Therefore, in an embodiment of the present invention the composition to be applied on the textile substrate to obtain a textile article containing graphene to produce a bandage to be applied on wounds, as defined above, is a water dispersion that comprises an effective quantity of a natural phytotherapeutic substance as defined above.

In the embodiment with binder consisting of a synthetic polymer, the composition comprises:
c) from 0.5 to 20% by weight of graphene;
d) from 10 to 40% by weight, preferably from 10 al 30% by weight, of a polymer binder;
b) from 1 to 10% by weight, preferably 3 to 8% by weight, of a compatibilizing solvent for said polymer binder;
c) from 0.1 to 2% by weight, preferably from 0.15 to 1.5% by weight, of a thickener;
d) from 0.1 to 10% by weight of a natural phytotherapeutic substance selected from the group consisting of curcumin, emodin, α-hederine and thymequinone; and
e) from 18 to 88.3% by weight of water.

In the embodiment with binder consisting of a polysaccharide, the composition comprises:
a) from 0.5 to 20% by weight of graphene;
b) from 1.0 to 50% by weight of polysaccharide;
c) from 0.1 to 10% by weight of a natural phytotherapeutic substance selected from the group consisting of curcumin, emodin, α-hederine and thymequinone; and
d) from 20 to 98.4% of water.

Preferably, the natural phytotherapeutic substance is present in an amount from 0.5 to 5% by weight, more preferably between 1 and 4% by weight.

With regard to the effectiveness of the bandage according to the invention in protecting the wound from bacterial, viral, fungal or yeast infections, the following standards are referred to. The standard UNI ISO 20743:2013 measures the antibacterial capacity of a textile product. The test is conducted in culture using a Petri dish, i.e., the sample of textile substrate treated and the untreated reference sample are placed in a culture broth and incubated at 37° C., with bacterial count before and after incubation. The test provides a number correlated to the antibacterial activity of the fabric. Typically, Staphylococcus aureus is used as gram-positive bacterial strain and Escherichia coli or Klebsiella as gram-negative bacterial strain. The result is expressed with the following values:
from 0 to 1: no antibacterial property
from 1 to 2: slight antibacterial property
from 2 to 3: significant antibacterial property
above 3: strong antibacterial activity.

The antibacterial activity according to the standard UNI ISO 20743:2013 of the bandage according to the present invention is greater than 1.

According to an aspect of the invention, the antibacterial activity is greater than 1.5. The standard UNI ISO 18184:2019 determines the antiviral activity of textile products. Without excluding the use of other viruses, the method usually employs Feline Calicivirus (VR-782) as enveloped virus and an influenza virus (H3N2 or H1N1) as non-enveloped virus. Lately, the virus responsible for the disease SARS-COV-2, or COVID-19, has also been used. According to the standard, the live cells that the virus infects in order to replicate must be observed.

The actual performances of the textile sample treated are determined by measuring the viral titer using a plate assay or the TCID₅₀ (Median Tissue Culture Infectious Dose) method. The result expresses the percentage reduction of viral activity with respect to the reference.

The antiviral activity of the bandage of the invention according to the standard ISO 18184:2019 is greater than 50%.

According to an aspect of the invention, the antiviral activity is greater than 60%.

### Methods of Applying the Graphene to the Textile Substrate

Preparation of the composition comprising graphene is preferably carried out by dispersing the binder in water in a receptacle stirred with a rotary blade stirrer, into which the graphene, the other components, and optionally the natural phytotherapeutic substance are then introduced. The composition is stirred until a uniform dispersion is obtained. Typically, stirring is conducted at a rotation speed of the stirrer ranging from 1000 to 2500 rpm for a time from 1 to 2 hours.

Various methods can be used to apply the composition comprising graphene to the textile substrate, such as printing with full coating of the substrate, impregnation of the substrate and spray coating of the substrate.

In both the full coating and the spray coating printing methods, the composition is applied on one side of the textile substrate and on one surface layer thereof, while in the impregnation method the composition can also be applied to the internal layers of the substrate.

Other application methods, known to those skilled in the art in the textile field, are also possible.

In the case in which the textile substrate is a film or a polymeric membrane, it is possible to incorporate the graphene in the polymer both before and after formation of the film, in the latter case for example by coating.

It is also possible to include the graphene between the layers of a multilayer textile substrate.

### Full Coating Printing

The graphene is dispersed in a print paste through the use of instruments that use high shear stresses in order to obtain a homogeneous dispersion.

The viscosity of the composition is in the range from 4000 to 30000 cPs and is mainly regulated by regulating the amount of thickener, or, in the case in which the binder is a polysaccharide, through the amount of this polysaccharide.

The viscosity of the composition is preferably in the range from 10000 to 20000.

### Spray Coating

The method consists of spraying a dispersion of graphene and binder on the textile substrate to obtain a light and uniform coating.

### Impregnation

This method consists of impregnating the textile substrate in a bath containing the composition containing the graphene in the form of dispersed nano-particles. The method allows the nano-particles to be deposited not only on the surface of the textile substrate but also between the inner fibers.

Application consists in immersing the textile substrate in an impregnation tank. The textile substrate is moved using rollers. After impregnation, the fabric is passed between two rubber coated rollers to eliminate the excess liquid.

### Post-Treatment

The textile article treated is then fed into an oven and heated at a temperature between 12° and 180° C. for a time between 1 and 10 minutes. The heat treatment causes evaporation of the water and of the compatibilizing solvent, if present, fixing of the binder and of the other components, and hardening of the composition.

### Incorporation of Graphene in a Microperforated Polymeric Film

A composition of polyurethane resin containing graphene suitable to obtain a microperforated membrane according to the present invention, and its preparation process, are described in the patent application WO 2018/202747 A1.

During the production process of the membrane, a degassing step is normally carried out to avoid the presence of bubbles inside the material. However, in the case of obtaining a microperforated membrane, degassing is not carried out, so that bubbles form, which during heating in the oven explode leaving voids. These voids, variable in size from 10 to 50 micrometers, are in fact the holes that pass through the membrane and allow the passage of air therethrough.

After obtaining the microperforated membrane, it is possible to laminate different fabrics on the two faces.

The textile article obtained with one of the aforesaid methods can advantageously be used to produce bandages for treating wounds.

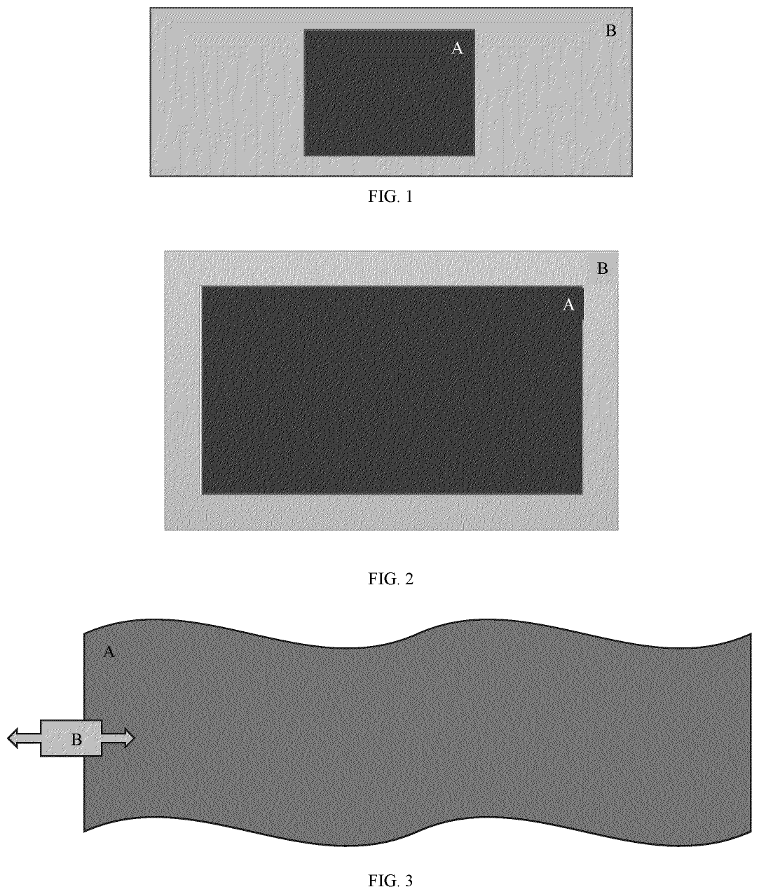

In an embodiment, the bandage according to the invention is provided with adhesive portions for fixing to the skin of the patient to be treated. Preferably, the adhesive portions are located at the edges of the bandage, as shown in FIG. 1.

A further feature of the bandages containing graphene according to the invention is their capacity to be sanitized making use of the excellent heat conduction capacities typical of graphene.

In fact, textile articles containing graphene can be sanitized using different technologies: gamma ray irradiation, thermal heating, also domestic, for example with an iron or hairdryer, and with IR irradiation. In all cases, it has been proved that the substrate treated is effectively sanitized of the substrate treated and the technical and antimicrobial properties are maintained.

The examples below illustrate some embodiments of the invention and are provided by way of non-limiting example.

### EXAMPLES

### Example 1

Nonwoven Spunlace Full Coating Printed with Print Paste Containing Graphene

A print paste containing graphene was prepared for full coating printing of a nonwoven spunlace with grammage 150 g/m², three-layer (outer layer with grammage of 50 g/m², consisting of polypropylene (PP) fibers-intermediate layer with grammage of 50 g/m². cellulose fibers-outer layer with grammage of 50 g/m²: PP fibers).
the print paste is prepared in a receptacle equipped with a mechanical stirrer (Dissolver DISPERMAT® CN100. Heavy Duty disk diameter 350 mm), in which the rotation speed is set to 200 rpm and the following are introduced:
100 kg of polymer binder consisting of water-based pre-catalyzed polyurethane resin containing around 30% of dry polymer (Resina E9010 marketed by CPL Chimica, Italy);
4 kg of graphene powder marketed by Directa Plus SpA with the brand name G+®, having a lateral dimension D90=4.7 μm and thickness=4.08 nm.
5 kg of compatibilizing solvent consisting of ethylene glycol (Sigma Aldrich);
0.4 kg of dispersant consisting of naphthalene sulfate (BASF);

A paste having a viscosity of 17234 cPs was obtained and stirring was taken to 1000 rpm and maintained for 5 hours.

The concentration of the graphene in the paste was 3.6% by weight.

The print paste was printed in a rotary machine using two cylinders so that an amount of print paste of 220 g/m² and an amount of graphene of 10 g/m² are applied on the fabric.

The printed fabric is placed in an oven and heated to 150° C. for 3 minutes to promote crosslinking of the polymer binder.

The fabric thus produced was analyzed to measure the following features:
surface resistivity (LORESTA GX): 4.3·10⁵Ω/□
in-plane thermal conductivity (HOT-DISK): 2.4 W/mK (ISO 22007-2)
infrared absorption: 90%
antibacterial activity (according to the standard UNI EN ISO 20743:2013):
   untreated fabric: for Klebsiella Pneumoniae (% of reduction of bacterial load) and for Staphylococcus Aureus (% of reduction of bacterial load)
   fabric printed with G+: 3.9 for Klebsiella Pneumoniae (99.987% reduction of bacterial load) and 4.3 for Staphylococcus Aureus (99.995% reduction of bacterial load)
antiviral activity (according to the standard UNI EN ISO 18184:2019):
   untreated fabric: 27% reduction of viral load of SARS-COV 2 virus
   fabric printed with G+: 97% reduction of viral load of SARS-COV 2 virus.

As shown in FIG. 1, the nonwoven thus printed was then cut into a rectangle 3×4 cm in size (A) and positioned on a plasticized strip with adhesive (B) attached, creating an adhesive plaster for medicating cuts and small wounds. The side of the nonwoven treated with graphene is maintained free to be able to come into contact with the skin, so that it can perform its function of cicatrizing and antimicrobial agent.

### Example 2

Biodegradable Nonwoven Full Coating Printed with Print Paste from Renewable Sources Containing Graphene

A composition was prepared consisting of an aqueous dispersion of graphene at the concentration of 4% by weight, with binder consisting of chitosan at the concentration of 4% by weight, and with the natural phytotherapeutic substance curcumin at the concentration of 2% by weight. Acetic acid was used to promote solubilization of the chitosan.

The print paste had the following composition:
water 225 g
acetic acid 2 g (1%)
chitosan 10 g (4%)
curcumin 5 g (2%)
graphene G+® 10 g (4%), lateral dimension D90=8 μm and thickness=5.44 nm.

The paste was printed on the whole of the surface of a three-layer nonwoven consisting of one layer of cellulose pulp inserted between two layers of bamboo viscose. Each layer of bamboo viscose had a grammage of 55 g/m², the layer of cellulose pulp had a grammage of 90 g/m² and the nonwoven as a whole had a grammage of 200 g/m².

Full coating printing allowed an amount of graphene of 10 g per square meter to be applied on the fabric.

After printing the nonwoven was treated at 150° C. for 3 minutes. After complete drying, surface resistivity and antibacterial activity were measured.

The surface electrical resistivity was measured with the method JIS K 7194, instrument Loresta-GX MCP-T700. The antibacterial activity was determined with the method UNI EN ISO 20743:2013.

The measurement results are set down in the table below:

| | |
|---|---|
| Surface resistivity (Ω/□) | Antibacterial activity |

| | | |
|---|---|---|
| 5.33 * 10⁵ | Klebsiella pneumoniae | 100% |
| | Staphylococcus aureus | 100% |

The textile article treated with chitosan and curcumin in addition to graphene exhibited an excellent antibacterial activity, allowing the bacterial load used in the test to be completely eliminated.

The nonwoven thus printed was then cut into a rectangle 9×5 cm in size (A), as shown in FIG. 2. A border of adhesive material was then positioned on the outer edge, allowing its use as adhesive plaster for medicating cuts and large wounds (B). The adhesive border was placed on the side of the nonwoven printed with graphene, so that the printed surface could be placed in contact with the skin.

In a different embodiment, the nonwoven was cut into strips 18×40 cm in size (A) to produce bandages to be wrapped around wounds which were then closed with a metal clip (B), as shown in FIG. 3. Also in this case, the side of nonwoven treated with graphene was positioned on the damaged skin, so that the graphene could perform its function of cicatrizing and antimicrobial agent.

### Example 3

Cotton Fabric Impregnated with Graphene

Composition of the fabric: 100% cotton. Weight: 110 g/m². Thickness: 193 μm.

The impregnation bath was prepared with the following composition:
water
13.5 g/L of graphene G+® lateral dimension D90=2.5 μm and thickness=3.06 nm
120 g/L polymer binder PADDING FM-N
30 g/L anti-migration agent SINERGIL N30

The impregnated fabric is then fed between a pair of pressing rollers to remove the excess impregnation bath, and then into an oven at a temperature between 100 and 200° C. to fix the impregnation composition on the fabric.

The quantity of bath absorbed by the sample during the impregnation process (pick-up) was 88% (88 g/m²), and the quantity of graphene fixed was 1.2 g/m².

The features of the fabric impregnated with graphene are:
surface resistivity after finishing: 3.35×10¹¹Ω/□
thermal conductivity (HOT DISK): 1.8 W/Mk (ISO 22007-2)
antibacterial activity (according to the standard UNI EN ISO 20743:2013):
   untreated fabric: 0.7 for Klebsiella Pneumoniae (80.047% reduction of microbial load) and 0.6 for Staphylococcus Aureus (74.881% reduction of microbial load)
   fabric impregnated with G+: >6.3 for Klebsiella Pneumoniae (100% reduction of microbial load) and 4.1 for Staphylococcus Aureus (99.992% reduction of microbial load)
antiviral activity (according to the standard UNI EN ISO 18184:2019):
   untreated fabric: 45% reduction of viral load of SARS-COV 2 virus
   fabric impregnated with G+: 98% reduction of viral load of SARS-COV 2 virus

The impregnated cotton thus prepared was cut into strips 18×40 cm in size (A) to produce bandages to wrap around wounds and to be closed, after medication, with a metal clip (B), as shown in FIG. 3. Also in this case, the side of the nonwoven treated with graphene was positioned on the damaged skin, so that the graphene could perform its function of cicatrizing and antimicrobial agent.

### Example 4

### Polyurethane Film Containing Graphene

A one layer polyurethane film with a thickness of 20 μm was prepared, to which graphene was added.

100 kg of polyurethane resin (ICAFLEX BR447 MATT3) was added to a receptacle equipped with mechanical stirrer (Dissolver DISPERMAT® CN100. Heavy Duty disk diameter 350 mm) and the rotation speed of the stirrer was set to 200 rpm. The following were then added:
5 kg of graphene powder G+® lateral dimension D90=17 μm and thickness=11.9 nm
0.8 kg of silica (OK 500)
2.7 kg of isocyanate catalyst (TRIXENE DP9B1376)
0.5 kg of flow additive (ADITEX LA 77)

A solvent consisting of dipropylene glycol methyl ether (Dowanol® DPM-Dow Chemical) was added to the above composition and the viscosity was set in the range 6,000-10,000 cPs. The blend was stirred at 1000 rpm for 1.5 hours.

The polyurethane composition was then spread evenly on release paper and placed in an oven heated to a temperature between 4° and 160° C., with formation of a polyurethane film.

Upon delivery from the oven, a flap of polyurethane film was detached by hand and then coupled to a cylinder to be rolled up.

A polyurethane film produced according to this formulation has an amount of graphene of 3 g/m².

Characterization of the film formed:
breathability: RET <10 m² Pa/W (ISO 31092)
impermeability: >1500 mm (ISO 20811)
abrasion resistance: >5000 cycles (ISO 12947)
surface resistivity: 7000Ω/□ (standard JIS K 7194)
in-plane thermal conductivity: 3.213 W/mK (ISO 22007-2)
IR absorbance: Absorption >90%
antibacterial properties (according to the standard UNI EN ISO 20743:2013):
   ref polyurethane film (without graphene): 2.9 for Klebsiella Pneumoniae (99.874% reduction of microbial load) and 1.8 for Staphylococcus Aureus (98.4% reduction of microbial load).
   polyurethane film with graphene: 3.9 for Klebsiella Pneumoniae (99.874% reduction of microbial load) and 3.8 for Staphylococcus Aureus (98.4% reduction of microbial load).

The polyurethane film thus prepared is then cut into a rectangle 3×4 cm in size (A) or larger and positioned on a plasticized strip with adhesive (B) attached, as shown in FIG. 1. The plaster thus prepared was used to medicate cuts and small wounds.

In an embodiment analogous to that of FIG. 2, the polyurethane film was cut into a rectangle 9×5 cm in size (A) and a border of adhesive material was positioned on the outer edge thereof, to allow its use as adhesive plaster for medicating cuts and large wounds (B).

1-11. (canceled)

12. A method comprising:
producing a bandage to be applied on wounds, utilizing a textile article comprising:
a textile substrate selected from a polymeric film, a fabric and a nonwoven; and
graphene, or a composition comprising graphene and a binding agent selected from a synthetic polymer and a polysaccharide;
wherein the graphene comprises nano-platelets in which at least 90% has a lateral dimension (x, y) from 500 to 50000 nm and a thickness (z) from 0.34 to 50 nm, and the textile article comprises from 0.01 to 20 g of the graphene per square meter.

13. The method of claim 12, wherein the graphene has a carbon-oxygen (C/O) ratio ≥30:1.

14. The method of claim 12, comprising dispersing the graphene in or on a polymeric film comprising the textile article.

15. The method of claim 12, comprising applying the composition comprising graphene and the binding agent selected from the synthetic polymer and the polysaccharide on the fabric or the nonwoven comprising the textile article.

16. The method of claim 12, wherein the composition comprises a natural phytotherapeutic substance with antimicrobial and antiviral properties.

17. The method of claim 16, wherein the composition comprises an effective quantity of the natural phytotherapeutic substance selected from a group consisting of curcumin, emodin, α-hederine, and thymequinone.

18. The method of claim 12, wherein at least 90% of the graphene nano-platelets have a lateral dimension (x, y) from 100 to 10000 nm.

19. The method of claim 12, wherein at least 90% of the graphene nano-platelets have a thickness (z) from 0.34 to 10 nm.

20. The method of claim 12, wherein the graphene has a C/O ratio ≥40:1.

21. The method of claim 19, wherein the graphene has a C/O ratio ≥70:1.

22. The method of claim 12, wherein the graphene contained in the textile article is present in an amount from 4 to 15 g of graphene per square meter.

23. The method of claim 12, wherein the bandage is self-adhesive.

24. A bandage to be applied on wounds, comprising a textile article further comprising:
a textile substrate selected from a polymeric film, a fabric and a nonwoven; and
graphene, or a composition comprising graphene and a binding agent selected from a synthetic polymer and a polysaccharide;
wherein the graphene comprises nano-platelets in which at least 90% has a lateral dimension (x, y) from 500 to 50000 nm and a thickness (z) from 0.34 to 50 nm, and the textile article comprises from 0.01 to 20 g of the graphene per square meter.

25. The bandage of claim 24, wherein the graphene has a carbon-oxygen (C/O) ratio ≥30:1.

26. The bandage of claim 24, wherein the composition comprises a natural phytotherapeutic substance with antimicrobial and antiviral properties.

27. The bandage of claim 26, wherein the composition comprises an effective quantity of the natural phytotherapeutic substance selected from a group consisting of curcumin, emodin, α-hederine, and thymequinone.

28. The bandage of claim 24, wherein at least 90% of the graphene nano-platelets have a lateral dimension (x, y) from 100 to 10000 nm.

29. The bandage of claim 24, wherein at least 90% of the graphene nano-platelets have a thickness (z) from 0.34 to 10 nm.

30. The bandage of claim 24, wherein the graphene contained in the textile article is present in an amount from 4 to 15 g of graphene per square meter.

31. The bandage of claim 24, wherein the bandage is self-adhesive.
